(19) United States
(12) Patent Application Publication
(10) Pub. No.: US 2011/0275705 A1
(43) Pub. Date: Nov. 10, 2011

(54) STABLE INJECTABLE OIL-IN-WATER DOCETAXEL NANOEMULSION

(75) Inventors: Gautam Vinod Daftary, Mumbai (IN); Srikanth Annappa Pai, Mumbai (IN); Mangesh Manikrao Kulkarni, Mumbai (IN)

(73) Assignee: BHARAT SERUMS AND VACCINES LTD., Mumbai (IN)

(21) Appl. No.: 13/055,613

(22) PCT Filed: Jul. 22, 2009
(86) PCT No.: PCT/IN09/00416

§ 371 Date: Apr. 25, 2011

(30) Foreign Application Priority Data
Jul. 23, 2008 (IN) 1565/MUM/2008

(51) Int. Cl. A61K 31/337; A61P 35/00; A61K 9/107

(52) U.S. Cl. 514/449

(57) ABSTRACT

The present invention describes Stable injectable oil-in-water Docetaxel nanoemulsion composition having Docetaxel concentrations as high as 20 mg/ml, devoid of hypersensitivity reaction and fluid retention. It employs Synthetic triglycerides, and DSPE PEG-2000, Natural phosphatides, Polyhydric alcohol and Water for injection. In another embodiment lyophilised products with added Cryoprotectants have been described which on reconstitution gives nanoemulsion suitable for parenteral administration.

### FIELD OF INVENTION

The present invention relates to oil-in-water nanoemulsion containing Docetaxel. The present invention particularly relates to a stable oil-in water nanoemulsion containing Docetaxel for parenteral administration

### BACKGROUND AND PRIOR ART

Docetaxel is commercially available in the form of an injection concentrate under brand name Taxotere and is indicated in the treatment of Breast Cancer, Non-small Cell Lung Cancer and Prostate Cancer. Taxotere is formulated in polysorbate 80 as solubiliser. Taxotere injection comprises two compartment formulations that require two-step dilution before infusion. The first step involves dilution with content of diluent vial (13% ethanol in water for injection) and the second step involves further dilution with diluents such as Dextrose Injection or normal saline etc. for parenteral administration.

Polysorbate 80 causes severe hypersensitivity reaction, and fluid retention, hence patients require pre-medications. Thus the marketed formulation has serious limitations with handling as well as side effects.

Further Polysorbate 80 can not be used with PVC delivery apparatus because of its tendency to leach diethyl hexyl phthalate, which is highly toxic.

To avoid these difficulties of mixing two solutions before injection following inventions have been reported

U.S. Pat. No. 5,478,860 describes a stable micro-emulsion composition comprising a mixture of an oil, a hydrophobic compound, and a polyethylene glycol-linked lipid, wherein the mixture is surrounded by a monolayer of a polar lipid. In one embodiment the mixture further includes phospholipids. In a preferred embodiment the hydrophobic compound is a therapeutic agent.

In one example it describes preparation of taxol (paclitaxel) emulsions. In this process taxol is first added to corn oil, and to it is added a mixture of MePEGS.2000-DSPE and EPC in chloroform; and then the chloroform is removed to get a thin film of lipids. This film is hydrated with HEPES buffered saline solution (pH 7.4); followed by addition of egg-phosphatidylcholine phospholipids-donating vesicles 70 nm in diameter. The mixture is passed through micro-emulsifier to give the micro-emulsion this indicates that the process goes through liposome formation.

US 2006/0067952A1 describes injectable oil-in-water emulsion of taxoid drugs, particularly, paclitaxel and docetaxel, comprising phospholipids and vegetable oils, which has to be diluted with aqueous fluid before administration.

A typical process for docetaxel emulsion comprises mixing docetaxel (0.05%), low oil (3.1%) (Soybean oil and additionally MCT oil), Egg lecithin (3.1%) and sufficient amount of Ethanol to form clear solution. The solution is dried under vacuum until residual ethanol is less than 2.0% by weight. Aqueous phase is prepared by dissolving glycerin (1.75) and glycine (0.5) in water. Aqueous phase is then added to oil phase under higher shear mixer to obtain crude emulsion. pH was adjusted to about 4-4.5 and the emulsion is passed through microfluidiser and the resulting emulsion is filtered through sterile 0.2μ filter.

We find that emulsion compositions described in US 2006/0067952A1 pertained to Paclitaxel except for one which describes Docetaxel. Paclitaxel and Docetaxel have stability at different pH i.e. Paclitaxel is more stable at pH around 7 and Docetaxel at pH around 4.5. Emulsions containing vegetable oils are highly unstable at acidic pH. Free fatty acids formation and coalescence of oil globules have been reported in such emulsions. Hence, the compositions described for Paclitaxel cannot be made applicable for Docetaxel without either adversely affecting the stability of Docetaxel or the emulsion stability as such.

Further composition of US 2006/0067952A1 describes stable compositions containing up to 0.5 mg/mL of the drug. However, to obtain higher drug content, the oil content has to be increased beyond 10% w/v. As concluded in this document itself “ . . . the emulsion formed are no longer acceptable as a safe parenteral drug delivery vehicle.” Hence, the compositions of US 2006/0067952A1 are not commercially viable if drug content required is more than 0.5 mg/mL.

WO2008/042841A2 describes pre-concentrate composition comprising docetaxel containing co-solvent like ethanol and propylene glycol, phospholipids, and pegylated phospholipids, suitable for parenteral administration to treat neoplasm conditions upon dilution with aqueous fluids. This pre-concentrate is a non-aqueous solution and forms emulsion on dilution. However when used in larger doses it may be harmful due to toxicity of solvents such as ethanol.

WO2008/042841A2 contains co-solvent which is harmful when given in larger doses.

Object

The principal object of the present invention is to make Docetaxel formulation which is devoid of hypersensitivity reaction and fluid retention there-by avoiding pre-medications.

Another object of the present invention is to avoid co-solvents like ethanol in the formulation thereby eliminating adverse effects that are caused by the cosolvents.

Yet another object of the present invention is to make stable Docetaxel formulation with higher levels of Docetaxel/ml of composition

Yet another object of the present invention is to make stable Docetaxel formulation that will give higher plasma concentrations of Docetaxel.

Yet another object of the present invention is to have Docetaxel formulation with increased stability and shelf life.

### SUMMARY OF THE INVENTION

Accordingly, the present invention provides a stable injectable oil-in-water Docetaxel nanoemulsion composition having pH 4.0-5.5, devoid of hypersensitivity reaction and fluid retention, comprising Docetaxel, Synthetic triglyceride oil, N-(carbonyl-methoxypolyethylene glycol 2000)-1,2-distearoyl-sn-glycero-3-phosphoethanolamine (DSPE PEG-2000), Purified natural phosphatides, Polyhydric alcohol and Water for injection.

The process for the preparation of these Docetaxel nanoemulsion composition comprises following steps

i) Docetaxel is dissolved in Synthetic triglyceride oil to get clear solution by sonication or heating forming the oil phase;

ii) Polyhydric alcohol is solubilised in Water for injection to form aqueous phase;

iii) N-(carbonyl-methoxypolyethylene glycol 2000)-1,2-distearoyl-sn-glycero-3-phosphoethanolamine is dispersed either in oil phase at step i or in aqueous phase at step ii or partly in aqueous phase in step i and partly in oily phase in step ii;

iv) purified natural phosphatide is dispersed in aqueous phase prepared at step ii;

v) the oil phase is added to aqueous phase under stirring to give a coarse emulsion;

vi) the coarse emulsion is homogenized to obtain the average globule size less than 200 nm, preferably less than 100 nm;

vii) pH of the emulsion obtained is adjusted to 4.0-5.5 either at step v or at step vi;

viii) the nanoemulsion obtained at the end of step vii, is filtered aseptically through 0.2μ filter and filled in vials under nitrogen.

In another embodiment of the present invention is provided a lyophilised composition for parenteral administration forming stable injectable oil-in-water Docetaxel nanoemulsion composition, having pH 4.0-5.5, on reconstitution, devoid of hypersensitivity reaction and fluid retention, comprising Docetaxel, Synthetic triglyceride oil, N-(carbonyl-methoxypolyethylene glycol 2000)-1,2-distearoyl-sn-glycero-3-phosphoethanolamine, Purified natural phosphatides, Polyhydric alcohol and cryoprotectants selected from Sucrose, Trehalose, Mannitol, Lactose or a mixture thereof.

The process for the preparation of these lyophilized Docetaxel nanoemulsion composition comprises following steps

i) Docetaxel is dissolved in Synthetic triglyceride oil to get clear solution by sonication or heating forming the oil phase;

ii) Polyhydric alcohol and Cryoprotectant are solubilised in Water for injection to form aqueous phase;

iii) N-(carbonyl-methoxypolyethylene glycol 2000)-1,2-distearoyl-sn-glycero-3-phosphoethanolamine is dispersed either in oil phase at step i or in aqueous phase at step ii or partly in aqueous phase in step i and partly in oily phase in step ii;

iv) purified natural phosphatide is dispersed in aqueous phase prepared at step ii;

v) the oil phase is added to aqueous phase under stirring to give a coarse emulsion;

vi) the coarse emulsion is homogenized to obtain the average globule size less than 200 nm, preferably less than 100 nm;

vii) pH of the emulsion obtained is adjusted to 4.0-5.5 either at step v or at step vi;

viii) the nanoemulsion obtained at the end of step vii, is filtered aseptically through 0.2μ filter, filled in vials and lyophilized.

### DETAIL DESCRIPTION OF THE INVENTION

Nanoemulsion

The definition of emulsions by the International Union of Pure and Applied Chemistry (IUPAC) states: “In an emulsion, liquid droplets and/or liquid crystals are dispersed in a liquid”. Obviously, microemulsions are excluded from this definition if the word “dispersed” is interpreted as non-equilibrium and opposite to “solubilized”, term that can be applied to microemulsions and micellar systems. Therefore, there is a fundamental difference between microemulsions and nano-emulsions. Microemulsions are equilibrium systems (i.e. thermodynamically stable), while nano-emulsions are non-equilibrium system with a spontaneous tendency to separate into the constituent phases. However, they are stabilized by addition of surfactants and other excipients.

According to this invention Nano-emulsions are emulsions (non-equilibrium systems) with a small droplet size (in the nanometer range, e.g. 20-200 nm).

Nanoemulsions are not to be mistaken with the classic “microemulsions”, which are thermodynamically stable and are often referred to as “self-emulsifying systems”. Microemulsions are formed when the surface tension is reduced to nearly zero and is only achieved by particular surfactants, combinations or particular packing of the adsorbed layer with surfactant and co-surfactant. These exhibit a very low viscosity and basically comprise swollen micelles with solubilized oil (and drugs). Microemulsion systems are transparent (optically isotropic), but upon dilution they can form conventional emulsion systems.

Nanoemulsion Composition of the Present Invention

The present invention describes nanoemulsions in two forms i) as liquid (nanoemulsions) and ii) as solid lyophilized powder (on reconstitution yielding nanoemulsion).

Docetaxel

Docetaxel used in the Examples is generally trihydrate and the concentration of Docetaxel in the nanoemulsion is 0.05%-2.0% w/v as expressed on anhydrous basis in liquid composition, preferably the concentration is 0.1%-2.0% w/v in the composition.

Synthetic Triglyceride Oil

After extensive experimentation, we find that nanoemulsions of Docetaxel using normal injectable oils do not have a good shelf life. The shelf life of the nanoemulsion made with mixtures of MCT oil and Vegetable oil is not satisfactory. Not bound by theory, we believe that there is interesterification and lipolysis reactions slowly deteriorating the stability of the nanoemulsions having vegetable oils. We have surprisingly found that such deterioration does not occur if we use synthetic triglycerides.

Medium chain triglyceride (MCT oil) is synthetically prepared using either natural source of glycerides or partly or totally synthetic materials. MCT are made from free fatty acid usually about 8 to about 12 carbon lengths. Representatives are commercially available as “Miglyol 840, MIGLYOL 812, CRODAMOL GTCC-PN, NEOBEE M-5 oil.

Synthetic triglyceride oil used in the nanoemulsion composition of the present invention is having fatty acids selected from Caproic acid, Caprylic acid, Capric acid, Lauric acid, Myristic acid, Oleic acid and mixtures thereof, preferably Caprylic acid is 50%-100% by weight, more preferably Caprylic acid is 85%-100% by weight.

The Synthetic triglyceride oil used in the present invention is selected from Medium chain triglyceride, Tricaprylin and Triolein and mixtures thereof.

Phosphatides

Phosphatides are used as emulsifier and also as a stabilizer for the nanoemulsion. Phosphatides used are either purified natural or synthetic phospholipids. Phospholipids are triester of glycerol with two fatty acid & one phosphate ion. The Purified natural phosphatides are selected from Purified Egg lecithin and Purified Soya lecithin and mixtures thereof.

Examples of synthetic Phospholipids include but not limited to phosphatidylcholine, Dipalmitoylphosphatidylcholine (DPPC), Distearoylphosphatidycholine (DSPC) and a mixture thereof.

Polyhydric Alcohols

The Polyhydric alcohol is selected from Glycerol, Propylene glycol and mixtures thereof.

Polyhydric alcohols are useful for preparing stable nanoemulsions.

DSPE PEG-2000 (Pegylated Distearoyl Phosphatidylethanolamine)

This is chemically known as N-(carbonyl-methoxypolyethylene glycol 2000)-1,2-distearoyl-sn-glycero-3-phosphoethanolamine. This acts like an emulsifier and stabiliser in the nanoemulsion of the present invention.

A phospholipid—PEG conjugate for this invention is PEG-phosphatidyl ethanolamine DSPE-PEG. The PEG chain in such phospholipid preferable has molecular weight in the range of 2000 to 5000. DSPE PEG-2000 is preferred.

While making the emulsions this DSPE PEG-2000 is added in aqueous phase or in oily phase or partly in aqueous and partly in oily phase.

Excipients

The composition of present invention may optionally contain pharmaceutically acceptable additives such as acidifier, alkalinizer, buffer, stabilizer, tonicity modifying agents and other biocompatible materials. Such agents are generally present in aqueous phase of emulsion which helps in stabilizing the emulsion.

Examples of acidifier are hydrochloric acid, citric acid, acetic acid, etc., but are not limited to these acids.

Examples of alkaliner include sodium hydroxide, sodium citrate etc.

Cryoprotectant materials such as Sucrose, Trehalose, Lactose, Mannitol are used to preserve the properties of nanoemulsion on Lyophilisation. Lyophilised product on reconstitution yields again nanoemulsion having similar specifications which was existing before Lyophilisation.

Other biocompatible materials include but are not limited to albumin, sorbitol, glycine, dextran etc.

In the nanoemulsion composition the ratio by weight of Synthetic triglyceride oil to Docetaxel is 1:1-100:1, preferably it is 10:1-50:1.

In the nanoemulsion composition the ratio by weight of Synthetic triglyceride oil to N-(carbonyl-methoxypolyethylene glycol 2000)-1,2-distearoyl-sn-glycero-3-phosphoethanolamine is 1:1-100:1, preferably 5:1-20:1.

In the nanoemulsion composition the ratio by weight of Synthetic triglyceride oil to Purified natural phosphatide is 4:1-40:1, preferably 7:1-20:1.

In the nanoemulsion composition the Polyhydric alcohol content is 0.5-3% w/v of the composition.

Lyophilised Nanoemulsion Composition

In the lyophilized nanoemulsion composition Docetaxel is 0.05%-2.0% w/v before Lyophilisation, preferably the concentration is 0.1%-2.0% w/v before Lyophilisation.

In the lyophilised nanoemulsion composition Synthetic triglyceride oil having fatty acids Caproic acid, Caprylic acid, Capric acid, Lauric acid, Myristic acid, Oleic acid and mixtures thereof, preferably Caprylic acid is 50%-100% by weight, more preferably Caprylic acid is 85%-100% by weight.

In the lyophilised nanoemulsion composition Synthetic triglyceride oil is selected from Medium chain triglyceride, Tricaprylin and Triolein and mixtures thereof.

In the lyophilised nanoemulsion composition the Purified natural phosphatides are selected from purified Egg lecithin and purified Soya lecithin and mixtures thereof.

In the lyophilised nanoemulsion composition Polyhydric alcohol is selected from Glycerol, Propylene glycol and mixtures thereof.

In the lyophilised nanoemulsion composition ratio by weight of Synthetic triglyceride oil to Docetaxel is 1:1-100:1, preferably 10:1-50:1.

In the lyophilised nanoemulsion composition ratio by weight of Synthetic triglyceride oil to N-(carbonyl-methoxypolyethylene glycol 2000)-1,2-distearoyl-sn-glycero-3-phosphoethanolamine is 1:1-100:1, preferably 5:1-20:1.

In the lyophilised nanoemulsion composition the ratio by weight of Synthetic triglyceride oil to Purified natural phosphatide is 4:1-40:1, preferably 7:1-20:1.

In the lyophilised nanoemulsion composition the Polyhydric alcohol content is 0.5-3% by weight.

In the lyophilised nanoemulsion composition the Sucrose content is upto 20% by weight.

### EXAMPLES

The invention will now be illustrated with the help of examples.

Examples are for illustrations purpose only and do not restrict the scope the invention.

Formulations of all Examples 1-20 and Example 28 are given in Table 1 (Page No. 26).

Observations of the samples of Examples 1 to Example 14 and Example 28 of nanoemulsions prepared are given in Table 2 (Page No. 27 and 28).

Stability results are given in Table 3 (Page No. 28).

After the Formulations of Examples 1-20 and Example 28, Examples of toxicity and other biological studies have been numbered Example No. 21 to Example No. 26. Example 27 provides shelf life data.

The materials used in these examples were of injectable grade/pharmaceutical grade and were procured locally.

Docetaxel trihydrate from Dr. Reddy's Laboratory.

Docetaxel anahydrous from Dabur Pharma Ltd.

Ethanol from Hayman.

MCT oil, Soya oil, DSPE PEG-2000 Sodium, Dipalmitoylphosphatidylcholine (DPPC), Egg lecithin, Sodium oleate from Lipoid.

Tricaprylin, Triolein, Sucrose, Trehalose from Sigma.

Glycerol from Qualigen.

Glycine from Merck.

Comparator sample Taxotere manufactured by Sanofi-Aventis is used in Examples whenever mentioned.

Equipments Used

Water bath, Ultra Turrax IKA stirrer, bath sonicator, Niro Soavi Homogenizer.

### Example 1

### Formula

| Ingredients | Quantity | |
|---|---|---|
| Docetaxel trihydrate | 214.0 | mg |
| Synthetic triglyceride oil (MCT oil) | 10.0 | gm |
| Egg Lecithin | 2.4 | gm |
| DSPE PEG-2000 | 1.0 | gm |
| Glycerol | 4.50 | gm |
| Water For injection | q.s to 200 | ml |

| | |
|---|---|
| 0.05N HCl Solution | q.s to adjust the pH |

The formulation composition of Example 1 is also given in Table 1.

Fatty acid composition of Synthetic triglyceride oil.

| Fatty acid composition | Example 1 |
|---|---|
| C₆ | 0.1% |
| C₈ | 54.7% |
| C₁₀ | 44.7% |
| C₁₂ | 0.3% |
| C₁₄ | Less than 0.1% |
| C_{18:1} | Nil |

The above Docetaxel nanoemulsion composition of Example 1 was prepared as follows:

Preparation of Oil Phase:
1. Docetaxel Trihydrate (214 mg) was added to MCT oil (10 g).
2. The above mixture was sonicated for 10 minutes and heated to about 70° C. and clear oily colorless liquid was obtained.

Preparation of Aqueous Phase
3. Glycerol (4.5 g) was mixed with Water for injection (qs to 200 ml) at Room Temperature (20° C.±5° C.).
4. DSPE PEG-2000 (1 g) was solubilized in above solution obtained in Step 3.
5. (2.4 g) Egg Lecithin was then dispersed in the aqueous solution obtained at Step 4.

Preparation of Coarse Emulsion
6. The oily phase is transferred to the aqueous phase under high speed stirring (on Ultra Turrax IKA stirrer) to obtain coarse emulsion.

Preparation of Nanoemulsion by Homogenization
7. The Coarse emulsion obtained was immediately passed through High Pressure Homogeniser and homogenized at 1200 bar for 5 minutes to get globule size distribution in 80-120 nm Range. Average globule size obtained was 99 nm.
8. The pH of the above emulsion was adjusted by the addition of dilute hydrochloric acid to 4.88.
9. Emulsion was then filtered through 0.2 g filter, filled in vials and sealed under nitrogen purging.

The pH and the particle size distribution of the composition was monitored during the process and the observations are given in Table 2. The particle size was monitored by Photon correlation spectroscopy method using Coulter Counter N4.

The stability of the nanoemulsion formed was examined by storing them at different temperatures. The results are given in Table 3.

### Example 2

### Comparative Example

The formulation composition is given in Table 1 and the Observations and stability results are given in Table 2 and Table 3 respectively.

Composition and process is same as Example 1 except that in Example 2 DSPE PEG-2000 was not used and homogenization is carried at higher pressure (1500 bar) for 20 minutes.

It was observed that it is not possible to reduce the average particle size below 140 m by increasing homogenization time for emulsion in the absence of pegylated phospholipids in the composition.

Further it is observed that the nanoemulsion is not stable in the absence of pegylated phospholipids. The samples of nanoemulsions of Example 2 shows settling of drug after 24 hrs where as emulsion product prepared incorporating pegylated phospholipids of example 1 dose not show any settling of drug at all storage conditions studied.

The examples of toxicity and other biological studies have been numbered after the 20 formulation examples. They are numbered Example No. 21 to Example No. 26.

Sample of docetaxel nanoemulsion of example was examined for toxicity, pharmacokinetic tests for plasma concentrations, using swiss albino mice and wistar rats. For comparison Taxotere was used. So also in vitro plasma studies of samples of example 1 and 2 were carried out.

### Example 21

### Acute Toxicity Study for Composition Product of Example 1

A) Single dose Acute Toxicity in Mice

Animal: Mice

Species: Swiss albino

No. of animals per group: 10

Dose: 150 mg/kg

| Sample | % mortality after 14 days |
|---|---|
| Example 1 | 50% |
| Taxotere | 100% |

### B) Single Dose Acute Toxicity in Rat

| | Mortality | | |
|---|---|---|---|
| Sample | 10 mg/kg | 30 mg/kg | 50 mg/kg |
| Example 1 | 0/6 | 0/6 | 2/6 |
| Taxotere | 0/6 | 2/6 | 5/6 |

### Example 22

### Toxicity Study for Composition Product of Example 1

Animal: Mice

Species: Swiss albino

Dose: 10, 22, 33, 50 mg/kg

Dosage schedule: q4d×3 (0, 4, 8 days)

| Sample | Dose | % mortality after 14 days |
|---|---|---|
| Example 1 | 10 mg/kg | 0% |
| | 22 mg/kg | 0% |
| | 33 mg/kg | 0% |
| | 50 mg/kg | 40% |
| Taxotere | 10 mg/kg | 0% |
| | 22 mg/kg | 10% |
| | 33 mg/kg | 20% |
| | 50 mg/kg | 70% |

### Example 23

### Comparative Single Dose Pharmacokinetic in Rat

Composition of Example 1 is used and Taxotere is used as a comparator.

Animal: Rat

Species: Wistar

Dose: 10 mg/kg

Plasma concentration (ng/mL)

| Time (hrs) | Taxotere | Example 1 |
|---|---|---|
| 0.083 | 1374.15 | 4070.84 |
| 0.5 | 445.41 | 564.21 |
| 4 | 166.29 | 221.33 |
| 8 | 59.11 | 191.82 |
| 24 | 68.49 | 53.74 |

Based on the graph obtained with plasma concentration in ng/mL (Y axis) plotted against time in hrs (X axis), it was found that Cₘₐₓ and AUC with composition of Example 1 were higher than that obtained with comparator product Taxotere.

### Example 24

### In-vitro Plasma Study of products of Example 1 and Example 2

Procedure
1. 0.2 ml of Docetaxel emulsion mixed in 0.9 ml of Human plasma in eppendorff tube.
2. Particle size of mixture is analyzed.
3. The mixed sample is incubated at 37° C. for 24 hr.
4. Particle size of incubated sample is analyzed.

Observations

| | | After Incubation at | |
|---|---|---|---|
| Example No. | Initial particle size | 37° C. for 24 Hr | |
| Example 1 | 105.1 nm | 106.2 | nm |
| | 105.1 nm | 103.9 | nm |
| Example 2 | 140 nm | 1.32 | micron |
| | 140 nm | 1.47 | micron |

Nanoemulsion prepared with pegylated phospholipid is stable in plasma where as emulsion prepared without pegylated phospholipid is not physically stable.

### Example 3

The process and quantities of ingredients are same as those used in Example 1 except that Docetaxel anhydrous was used in place of Docetaxel trihydrate.

The formulation composition is given in Table 1 and the Observations and stability results are given in Table 2 and Table 3 respectively.

Conclusion

This example shows emulsion with docetaxel anhydrous shows similar stability profile as docetaxel trihydrate.

### Example 4

### Nanoemulsion Prepared Using Mixture of Vegetable Oil and MCT Oil (This Example is not of Invention)

The formulation composition is given in Table 1.

Procedure

Same as of Example 1 with appropriate ingredients and their weights as in the formulations.

Observations and stability results are given in Table 2 and Table 3 respectively. Though the emulsion was stable in 24 hour test, the physical stability was not found satisfactory on storage for longer period: that is separation of oil layer was observed. The free fatty acid content also increased significantly on storage for 3 months at 25° C., the product was rancid perhaps because of soy oil and aqueous contact at low pH.

### Example 5

### Prepared as Per the Composition and Process of US 2006/0067952A1—Comparative Example

The formulation composition is given in Table 1.

Observations and stability results are given in Table 2 and Table 3 respectively.

Settling of the drug in 24 hours was observed and does not form a stable emulsion. This is perhaps because of the composition ethanol, soya oil, and not containing DSPE PEG-2000.

### Example 6

### In this Example the Formulation was Prepared with DPPC as Surfactant Instead of Egg Lecithin

The formulation composition is given in Table 1.

Procedure

Same as Example 1 with appropriate ingredients and their weights as in the formulations.

Instead of egg lecithin DPPC was dispersed in aqueous phase.

Observations and stability results are given in Table 2 and Table 3 respectively.

### Example 7

### This Formulation was Prepared with 7% of MCT Oil

The formulation composition is given in Table 1.

Procedure

Same as Example 1 with appropriate ingredients and their weights as in the formulations.

Observations and stability results are given in Table 2 and Table 3 respectively.

### Example 8

### This Formulation was Prepared with 10% of MCT Oil

The formulation composition is given in Table 1.

Procedure

Same as Example 1 with appropriate ingredients and their weights as in the formulations.

Observations and stability results are given in Table 2 and Table 3 respectively.

### Example 9, 10

### These Formulations are Similar to Each Other Except for Different Concentrations of DSPE PEG-2000

Pharmacokinetics study details on Example 9 and 10 are provided in Example 25. Antitumor efficacy study details on Example 9 and 10 are provided in Example 26.

The formulation composition is given in Table 1.

Procedure of Examples 9 and 10

Same as Example 1 with appropriate ingredients and their weights as in the formulations.

Observations are given in Table 2.

The Stability of the products of Example 9 and Example 10 were found to be good and both being similar, product of Example 10 was taken for shelf life study as described in Example 27. Shelf life results are given in Table 4 and Table 5 and found to be satisfactory.

### Example 25

### Pharmacokinetic Study for Composition Product of Example 9 and Example 10

Plasma samples were analysed by HPLC method. Details of HPLC methods are given below:
Column: C-18 (100×4.6 mm×3μ
Column temp.: 60° C.
Flow rate: 1 mL/min.
Mobile phase: Methanol:THF:Water:Ammonium hydroxide (60:2.5:37.5:0.1). Adjust the pH with Formic acid to 6.0
Wave length: 230λ

Animal: Rat

Species: Wistar

Dose: 10 mg/kg

| | Plasma concentration (ng/mL) | | |
|---|---|---|---|
| Time (hrs) | Taxotere | Example 9 | Example 10 |
| 0.25 | 1128.5 | 7007.5 | 8881.4 |
| 0.5 | 728.4 | 1620.35 | 2011 |
| 1 | 557.95 | 943.05 | 858.3 |
| 3 | 450.85 | 425.85 | 420.9 |
| 4 | 425.85 | 497.8 | 444.65 |
| 6 | 461.8 | 469.3 | 560.4 |
| 8 | 582.3 | 601.35 | 576.45 |

Above data indicate that approximately 8 times higher concentration of docetaxel is available in plasma compared to conventional preparation of Docetaxel i.e. Taxotere.

### Example 26

### Antitumor Efficacy of Samples of the Product of Example 10

Antitumor efficacy was evaluated in SCID mice inducing MX-1 tumors. The drug was injected at 8.5 mg/kg and 17 mg/kg three times at four day intervals (q4d).

### Comparative Tumor Volume Reduction Data for Example 10 & Taxotere in SCID Mice Having MX-1 Tumors

| | Relative Tumor volume | | | | | |
|---|---|---|---|---|---|---|
| | | | Example 10 | | Taxotere | |
| | | | Dose - | Dose - | Dose - | Dose - |
| | | V. | 25.5 | 51 | 25.5 | 51 |
| Day | Control^{#} | Control^{##} | mg/kg^{@} | mg/kg^{@} | mg/kg^{@} | mg/kg^{@} |
| 1 | 1 | 1 | 1 | 1 | 1 | 1 |
| 3 | 2.52 | 1.75 | 1.48 | 1.27 | 1.64 | 1.08 |
| 5 | 3.91 | 2.11 | 1.46 | 1.36 | 1.68 | 1.51 |
| 7 | 5.57 | 3.75 | 1.56 | 0.70 | 1.24 | 0.69 |
| 9 | 6.77 | 4.61 | 1.01 | 0.51 | 0.94 | 0.54 |
| 11 | 8.72 | 5.88 | 0.63 | 0.33 | 0.61 | 0.34 |
| 13 | 10.11 | 7.32 | 0.33 | 0.12 | 0.25 | 0.12 |
| 15 | 12.54 | 10.29 | 0.17 | 0.08 | 0.18 | 0.07 |

^{#}Untreated group
^{##}Untreated vehicle control (without docetaxel) group
^{@}Total dose administered by intravenous route in three divided dose q4d (every four days) × 3

Above data conclusively shows antitumor efficacy of new invented formulation.

### Example 11

### Formulation Prepared with Sodium Oleate

The formulation composition is given in Table 1. Sodium oleate is incorporated in the aqueous phase.

Procedure

Same as Example 1 with appropriate ingredients and their weights as in the formulations.

Observations and stability results are given in Table 2 and Table 3 respectively.

### Example 27

### Shelf Life Study

Product of composition Example 10 was studied for stability. Results of stability are shown in Table 4 and Table 5. Data provided in Table 4 indicates the composition is stable at 2-8° C. for the 6 month time period studied.

TABLE 4
Stability Data of 2-8° C.

| Tests | Initial | 2M | 3M | 6M |
|---|---|---|---|---|
| Appearance | WOL | WOL | WOL | WOL |
| pH | 4.94 | 5.08 | 4.87 | 4.54 |
| Particle Size (nm) | 97.0 | 107.5 | 109.1 | 109.0 |
| Docetaxel content | 1.026 | 1.025 | 1.030 | 1.00 |

WOL—White opaque liquid

TABLE 5
Stability Data of 25° C.

| Tests | Initial | 1M | 2M | 3M |
|---|---|---|---|---|
| Appearance | WOL | WOL | WOL | WOL |
| pH | 4.94 | 4.09 | 3.84 | 3.56 |
| Particle Size (nm) | 97.0 | 107.6 | 112.4 | 127.1 |
| Docetaxel content | 1.026 | 0.992 | 0.964 | 0.883 |

WOL—White opaque liquid

### Example 12-14, 28

### Nanoemulsion Made with Synthetic Triglycerides Oils of Different Compositions Prepared Using MCT Oil, Tricaprylin, Triolein

### Fatty Acid Composition of Synthetic Triglyceride Oil Used in the Examples 12-14 and Example 28

| Fatty acid | Example | Example | Example | Example |
|---|---|---|---|---|
| composition | 12 | 13 | 14 | 28 |
| C₆ | Less than 0.1% | Less than 0.1% | Nil | Nil |
| C₈ | 94.34% | 92.73% | 100% | 100% |
| C₁₀ | 5.58% | 2.235% | Nil | Nil |
| C₁₂ | Less than 0.1% | Less than 0.1% | Nil | Nil |
| C₁₄ | Less than 0.1% | Less than 0.1% | Nil | Nil |
| C_{18:1} | Nil | 5% | Nil | Nil |

Formulations are given in Table 1.

Procedure

Same as Example 1 with appropriate ingredients and their weights as in the formulations.

Observations and stability results are given in Table 2 and Table 3 respectively. These examples show the preparation of stable nanoemulsions with higher levels of docetaxel.

Examples 15-20 are for illustration of second embodiment of the present invention wherein the nanoemulsion is lyophilized and that can be reconstituted back to nanoemulsion and they do not limit the scope of the invention.

### Examples 15-20

### Lyophilised Formulations

Procedure has been described in text but is basically same as that of Example 1 with appropriate ingredients and their weights as in the formulations, except that Cryoprotectant like Sucrose, Trehalose is added to aqueous phase. After adjusting the pH, product is filtered through 0.2μ sterile filter & 5 mL was filled in each vial. All vials lyophilized using following conditions:

Freezing temperature: −45° C. for 240 min.

Primary drying temperature: 5° C.

Primary drying time: 52-60 hrs

Primary drying vacuum −100 mTorr

Secondary drying temperature −25° C.

Secondary drying time −12 hrs

Secondary drying vacuum −50 mTorr

All Lyophilized cake reconstituted with 5 ml of water for injection except lyophilized cake from Example 19 reconstituted with 15 ml of water for injection. Observations and shelf life studies by examination of nanoemulsions on reconstitution of the lyophilized product stored at 2-8° C. are given in Table 6 and Table 7 respectively. The stability is found to be satisfactory.

TABLE 6
Observations on Example 15-20

| Observations | Example 15 | Example 16 | Example 17 | Example 18 | Example 19 | Example 20 |
|---|---|---|---|---|---|---|
| Appearance | White cake | White cake | White cake | White cake | White cake | White cake |
| Docetaxel content | 1.08 mg/ml | 1.0 mg | 0.98 mg/ml | 1.02 mg/ml | 0.97 mg/ml | 5.02 mg/ml |
| pH on reconstitution | 4.8 | 5.2 | 4.96 | 4.87 | 4.8 | 4.90 |
| Particle size - | 102 nm | 110 nm | 85 nm | 96 nm | 115 nm | 110 nm |
| before lyophilisation | | | | | | |
| Particle size- | 112 nm | 102 nm | 95 nm | 98 nm | 137 nm | 108 nm |
| after lyophilisation | | | | | | |

TABLE 7
Stability data - 2-8° C.

| | Example 15 | | | Example 16 | | | Example 17 | | |
|---|---|---|---|---|---|---|---|---|---|
| Tests | 1M | 2M | 3M | 1M | 2M | 3M | 1M | 2M | 3M |
| Docetaxel content (mg/ml) | 1.08 | 1.06 | 1.08 | 1.0 | 0.99 | 0.97 | 0.97 | 0.98 | 0.96 |
| pH on reconstitution | 4.7 | 4.75 | 4.65 | 5.1 | 5.0 | 5.1 | 4.95 | 4.9 | 4.95 |
| Particle sixe (nm) | 110 | 112 | 108 | 105 | 100 | 98.2 | 98 | 102 | 100 |
| (On reconstitution) | | | | | | | | | |

| | Example 18 | | | Example 19 | | | Example 20 | | |
|---|---|---|---|---|---|---|---|---|---|
| Tests | 1M | 2M | 3M | 1M | 2M | 3M | 1M | 2M | 3M |
| Docetaxel content (mg/ml) | 1.0 | 1.01 | 1.0 | 0.98 | 0.97 | 0.96 | 5.01 | 4.98 | 4.97 |
| pH on reconstitution | 4.8 | 4.8 | 4.75 | 4.8 | 4.85 | 4.75 | 4.8 | 4.60 | 4.75 |
| Particle sixe (nm) | 97 | 92 | 98 | 132 | 125 | 130 | 108 | 109 | 112 |
| (On reconstitution) | | | | | | | | | |

### ADVANTAGES OF THE INVENTION

1. The compositions of the present invention are free from ethanol and surfactant Polysorbate-80. Therefore composition of present invention is devoid of hypersensitivity reaction and fluid retention characteristics of these ingredients.
2. The process of preparation is free from any solvent and co-solvent like ethanol and chloroform.
3. No pre-medication required to overcome hypersensitivity reactions experienced with currently marketed preparation.
4. Higher Cₘₐₓ and AUC would lead to better efficacy at equivalent doses. Alternatively equivalent therapeutic efficacy could be obtained at lower doses which in turn would reduce toxic effects of the drug.
5. Process gives stable nanoemulsion which gives Enhanced Permeability Retention (EPR) effect.
6. The nanoemulsions of the present invention are stable for longer period and commercially viable.
7. The nanoemulsions of the present invention are having higher strength of docetaxel and higher plasma concentrations.

TABLE 1
Docetaxel Nanoemulsion Compositions Prepared in Examples 1-20 and Example 28

| | Examples | | | | | | | | | |
|---|---|---|---|---|---|---|---|---|---|---|
| Ingredients | 1 | 2* | 3 | 4* | 5* | 6 | 7 | 8 | 9 | 10 |
| Docetaxel trihydrate mg | 214 | 214 | — | 107 | 53.5 | 108 | 108 | 108 | 108 | 108 |
| Docetaxel anhydrous mg | | | 214 | | | | | | | |
| Ethanol ml | | | | | 2 | | | | | |
| MCT Oil g | 10 | 10 | 10 | 2.5 | 1.5 | 5.0 | 7.0 | 10 | 5.0 | 5.0 |
| Tricaprylin g | | | | | | | | | | |
| Triolein g | | | | | | | | | | |
| Soya oil g | | | | 2.5 | 1.5 | | | | | |
| Na Oleate mg | | | | | | | | | | |
| Egg lecithin g | 2.4 | 2.4 | 2.4 | 1.2 | 3.1 | | 1.2 | 1.2 | 1.2 | 1.2 |
| DSPE PEG-2000 g | 1.0 | — | 1.0 | 0.5 | — | 1.0 | 1.0 | 1.0 | 0.75 | 1.0 |
| DPPC g | | | | | | 1 | | | | |
| Glycerol g | 4.50 | 4.50 | 4.50 | 2.25 | 2.25 | 2.25 | 2.25 | 2.25 | 2.25 | 2.25 |
| Glycine mg | | | | | 50 | | | | | |
| Sucrose g | | | | | | | | | | |
| Trehalose g | | | | | | | | | | |
| Water ml qs to | 200 | 200 | 200 | 100 | 100 | 100 | 100 | 100 | 100 | 100 |
| pH | 4.88 | 4.63 | 4.94 | 4.50 | 4.68 | 4.75 | 4.88 | 4.73 | 4.80 | 4.92 |

| | Examples | | | | | | | | | | |
|---|---|---|---|---|---|---|---|---|---|---|---|
| Ingredients | 11 | 12 | 13 | 14 | 15 | 16 | 17 | 18 | 19 | 20 | 28 |
| Docetaxel trihydrate mg | 108 | 1000 | 1000 | 1000 | 108 | 108 | 108 | 108 | 324 | 500 | 2000 |
| Docetaxel anhydrous mg | | | | — | | | | | | | |
| Ethanol ml | | | | — | | | | | | | |
| MCT Oil g | 5.0 | 1 | 0.5 | — | 5.0 | 5.0 | 5.0 | 5.0 | 15 | 2 | |
| Tricaprylin g | | 7 | 9 | 10 | | | | | | 4 | 20 |
| Triolein g | | | 0.5 | — | | | | | | | |
| Soya oil g | | | | — | | | | | | | |
| Na Oleate mg | 30 | | | — | | | | | | | |
| Egg lecithin g | 1.2 | 1.2 | 1.2 | 1.2 | 1.2 | 1.2 | 1.2 | 1.2 | 1.2 | 1.2 | 1.2 |
| DSPE PEG-2000 g | 1.0 | 1.0 | 1.0 | 1.0 | 1.0 | 1.0 | 1.0 | 1.0 | 1.0 | 1.0 | 1.0 |
| DPPC g | | | | — | | | | | | | |
| Glycerol g | 2.25 | 2.25 | 2.25 | 2.25 | 2.25 | 2.25 | 2.25 | 2.25 | 2.25 | 2.25 | 2.25 |
| Glycine mg | | | | — | | | | | | | |
| Sucrose g | | — | — | — | 10 | | 5 | 15 | 20 | 10 | |
| Trehalose g | | | | — | | 5 | | | | | |
| Water ml qs to | 100 | 100 | 100 | 100 | 100 | 100 | 100 | 100 | 100 | 100 | 100 |
| pH | 4.73 | 4.90 | 4.80 | 4.72 | 4.80 | 5.20 | 4.96 | 4.87 | 4.80 | 4.90 | 4.80 |

*Not of invention

TABLE 2
Observations of the samples of Examples 1 to Example 14 and Example 28 of nanoemulsions prepared

| | | Observations | | | | | |
|---|---|---|---|---|---|---|---|
| Tests | | Example 1 | Example 2 | Example 3 | Example 4 | Example 5 | Example 6 |
| Appearance | | White Opaque | White Opaque | White Opaque | White Opaque | White Opaque | White Opaque |
| | | Liquid | Liquid | Liquid | Liquid | Liquid | Liquid |
| pH | Aq. Phase | 5.65 | 6.33 | 6.59 | 6.69 | 7.8 | 6.6 |
| | Coarse | 5.82 | 5.98 | 6.11 | 7.01 | 7.2 | 6.15 |
| | Final Homogenisation | 5.68 | 5.96 | 6.08 | 6.64 | 6.34 | 5.79 |
| | After pH adjustment | 4.88 | 4.63 | 4.94 | 4.50 | 4.68 | 4.75 |
| Particle | Coarse | 212.0 | 283.4 | 164.7 | 201.6 | 180.0 | 256.2 |
| Size (nm) | Final Homogenisation | 95.9 | 142.2 | 94.8 | 101.8 | 99.0 | 106.8 |
| | After pH adjustment | 99.2 | 140.7 | 93.0 | 102.4 | 99.7 | 104.1 |

| | | Observations | | | | |
|---|---|---|---|---|---|---|
| Tests | | Example 7 | Example 8 | Example 9 | Example 10 | Example 11 |
| Appearance | | White Opaque | White Opaque | White Opaque | White Opaque | White Opaque |
| | | Liquid | Liquid | Liquid | Liquid | Liquid |
| pH | Aq. Phase | 5.5 | 5.56 | 6.0 | 5.90 | 8.1 |
| | Coarse | 5.82 | 5.98 | 6.34 | 6.20 | 7.18 |
| | Final Homogenisation | 5.62 | 5.68 | 6.59 | 5.60 | 7.15 |
| | After pH adjustment | 4.88 | 4.73 | 4.80 | 4.92 | 4.73 |
| Particle | Coarse | 222.0 | 253.4 | 188 | 170 | 190 |
| Size (nm) | Final Homogenisation | 112.0 | 111.3 | 104 | 98 | 108 |
| | After pH adjustment | 111.2 | 112.0 | 104.7 | 96.20 | 102 |

| | | Observations | | | |
|---|---|---|---|---|---|
| Tests | | Example 12 | Example 13 | Example 14 | Example 28 |
| Appearance | | White Opaque | White Opaque | White Opaque | White Opaque |
| | | Liquid | Liquid | Liquid | Liquid |
| pH | Aq. Phase | 6.2 | 6.25 | 6.2 | 5.80 |
| | Coarse | 5.82 | 5.80 | 5.65 | 5.82 |
| | Final Homogenisation | 5.72 | 5.70 | 5.65 | 5.40 |
| | After pH adjustment | 4.90 | 4.80 | 4.72 | 4.80 |
| Particle | Coarse | 202 | 198 | 212 | 228 |
| Size (nm) | Final Homogenisation | 102 | 110 | 98 | 112 |
| | After pH adjustment | 103 | 105 | 102 | 108 |

TABLE 3
Stability Results

| Example | | Temperature Conditions | | |
|---|---|---|---|---|
| No. | Time | 2-8° C. | 25° C. | 40° C. |
| 1 | Initial | White Opaque Liquid | White Opaque Liquid | White Opaque Liquid |
| | 24 hrs | White Opaque Liquid | White Opaque Liquid | White Opaque Liquid |
| 2 | Initial | White Opaque Liquid | White Opaque Liquid | White Opaque Liquid |
| | 24 hrs | White opaque liquid | White opaque liquid | White opaque liquid |
| | | with settling of drug | with settling of drug | with settling of drug |
| 3 | Initial | White Opaque Liquid | White Opaque Liquid | White Opaque Liquid |
| | 24 hrs | White Opaque Liquid | White Opaque Liquid | White Opaque Liquid |
| 4 | Initial | White Opaque Liquid | White Opaque Liquid | White Opaque Liquid |
| | 24 hrs | White Opaque Liquid | White Opaque Liquid | White Opaque Liquid |
| 5 | Initial | White Opaque Liquid | White Opaque Liquid | White Opaque Liquid |
| | 24 hrs | White opaque liquid | White opaque liquid | White opaque liquid |
| | | with settling of drug | with settling of drug | with settling of drug |
| 6 | Initial | White Opaque Liquid | White Opaque Liquid | White Opaque Liquid |
| | 24 hrs | White Opaque Liquid | White Opaque Liquid | White Opaque Liquid |
| 7 | Initial | White Opaque Liquid | White Opaque Liquid | White Opaque Liquid |
| | 24 hrs | White Opaque Liquid | White Opaque Liquid | White Opaque Liquid |
| 8 | Initial | White Opaque Liquid | White Opaque Liquid | White Opaque Liquid |
| | 24 hrs | White Opaque Liquid | White Opaque Liquid | White Opaque Liquid |
| 11 | Initial | White Opaque Liquid | White Opaque Liquid | White Opaque Liquid |
| | 24 hrs | White Opaque Liquid | White Opaque Liquid | White Opaque Liquid |
| 12 | Initial | White Opaque Liquid | White Opaque Liquid | White Opaque Liquid |
| | 24 hrs | White Opaque Liquid | White Opaque Liquid | White Opaque Liquid |
| 13 | Initial | White Opaque Liquid | White Opaque Liquid | White Opaque Liquid |
| | 24 hrs | White Opaque Liquid | White Opaque Liquid | White Opaque Liquid |
| 14 | Initial | White Opaque Liquid | White Opaque Liquid | White Opaque Liquid |
| | 24 hrs | White Opaque Liquid | White Opaque Liquid | White Opaque Liquid |
| 28 | Initial | White Opaque Liquid | White Opaque Liquid | White Opaque Liquid |
| | 24 hrs | White Opaque Liquid | White Opaque Liquid | White Opaque Liquid |

1. Stable injectable oil-in-water Docetaxel nanoemulsion composition having droplet size less than 200 nm and pH 4.0-5.5, devoid of hypersensitivity reaction and fluid retention, comprising Docetaxel; synthetic triglyceride oil as the only oil component; N-(carbonyl-methoxypolyethylene glycol)-1,2-distearoyl-sn-glycero-3-phosphoethanolamine in which the PEG chain has a molecular weight of 2000 to 5000 and purified natural phosphatide as the only emulsifiers; glycerol; and water for injection and free from any further solvent or co-solvent.

2. A composition as claimed in claim 1 wherein the N-(carbonyl-methoxypolyethylene glycol)-1,2-distearoyl-sn-glycero-3-phosphoethanolamine is N-(carbonyl-methoxypolyethylene glycol 2000)-1,2-distearoyl-sn-glycero-3-phosphoethanolamine.

3. Lyophilised composition for parenteral administration forming stable injectable oil-in-water Docetaxel nanoemulsion composition, having droplet size less than 200 nm and pH 4.0-5.5, on reconstitution, devoid of hypersensitivity reaction and fluid retention, comprising Docetaxel, synthetic triglyceride oil as the only oil component; N-(carbonyl-methoxypolyethylene glycol)-1,2-distearoyl-sn-glycero-3-phosphoethanolamine in which the PEG chain has a molecular weight of 2000 to 5000 and purified natural phosphatide as the only emulsifiers; glycerol; and cryoprotectant and free from any further solvent or co-solvent.

4. A composition as claimed in claim 3 wherein the N-(carbonyl-methoxypolyethylene glycol)-1,2-distearoyl-sn-glycero-3-phosphoethanolamine is N-(carbonyl-methoxypolyethylene glycol 2000)-1,2-distearoyl-sn-glycero-3-phosphoethanolamine.

5. A composition as claimed in claim 1 wherein Docetaxel is 0.05%-2.0% w/v of the composition.

6. A composition as claimed in claim 1 wherein the synthetic triglyceride oil has fatty acids selected from caproic acid, caprylic acid, capric acid, lauric acid, myristic acid, Oleic acid and mixtures thereof.

7. A composition as claimed in claim 1 wherein the synthetic triglyceride oil has caprylic acid 85%-100% by weight.

8. A composition as claimed in claim 1 wherein the synthetic triglyceride oil is selected from medium chain triglyceride, tricaprylin and triolein and mixtures thereof.

9. A composition as claimed in claim 1 wherein the purified natural phosphatides are selected from purified egg lecithin and purified soya lecithin and mixtures thereof.

10. A composition as claimed in claim 1 wherein the ratio by weight of synthetic triglyceride oil to Docetaxel is 1:1-100:1.

11. A composition as claimed in claim 1 wherein ratio by weight of synthetic triglyceride oil to Docetaxel is 10:1-50:1.

12. A composition as claimed in claim 2 wherein the ratio by weight of synthetic triglyceride oil to N-(carbonyl-methoxypolyethylene glycol 2000)-1,2-distearoyl-sn-glycero-3-phosphoethanolamine is 1:1-100:1.

13. A composition as claimed in claim 2 wherein ratio by weight of synthetic triglyceride oil to N-(carbonyl-methoxypolyethylene glycol 2000)-1,2-distearoyl-sn-glycero-3-pphosphoethanolamine is 5:1-20:1.

14. A composition as claimed in claim 1 wherein the ratio by weight of synthetic triglyceride oil to purified natural phosphatide is 4:1-40:1.

15. A composition as claimed in claim 1 wherein the ratio by weight of synthetic triglyceride oil to purified natural phosphatide is 7:1-20:1.

16. A composition as claimed in claim 1 wherein the glycerol content is 0.5-3% w/v of the composition.

17. A process for the preparation of Docetaxel nanoemulsion composition as claimed in claim 1 comprising following steps
i) dissolving Docetaxel in the synthetic triglyceride oil to get clear solution by sonication or heating forming the oil phase;
ii) solubilising glycerol is solubilised in water for injection to form aqueous phase;
iii) dispersing the N-(carbonyl-methoxypolyethylene glycol)-1,2-distearoyl-sn-glycero-3-phosphoethanolamine in the oil phase at step or in the aqueous phase at step ii, or partly in the aqueous phase in step i and partly in the oily phase in step
iv) dispersing purified natural phosphatide in the aqueous phase prepared at step
v) adding the oil phase to the aqueous phase under stirring to give a coarse emulsion:
vi) homogenizing the coarse emulsion to obtain the average globule size of less than 200 nm, preferably less than 100 nm;
vii) adjusting the pH of the emulsion obtained to 4.0-5.5 either at step v or at step vi; and
viii) filtering the nanoemulsion obtained at the end of step vii aseptically through 0.2μ filter and filled in vials under nitrogen.

18. A Docetaxel nanoemulsion composition prepared by a process as claimed in claim 17.

19. Lyophilised composition as claimed in claim 3 wherein the cryoprotectant is selected from sucrose, trehalose, mannitol, lactose or a mixture thereof.

20. Lyophilised composition as claimed in claim 3 wherein the cryoprotectant is Sucrose in an amount up to 20% by weight.

21. A process for the preparation of lyophilized composition as claimed in claim 3 comprising following steps
i) dissolving Docetaxel in the Synthetic triglyceride oil to get clear solution by sonication or heating forming the oil phase;
ii) solubilising glycerol and the cryoprotectant in water for injection to form aqueous phase;
iii) dispersing N-(carbonyl-methoxypolyethylene glycol)-1,2-distearoyl-sn-glycero-3-phosphoethanolamine in the oil phase at step I, or in the aqueous phase at step ii, or partly in the aqueous phase in step i and partly in the oily phase in step ii;
iv) dispersing purified natural phosphatide in the aqueous phase prepared at step ii;
v) adding the oil phase to the aqueous phase under stirring to give a coarse emulsion;
vi) homogenizing the coarse emulsion to obtain the average globule size of less than 200 nm, preferably less than 100 nm;
vii) adjusting the pH of the emulsion obtained to 4.0-5.5 either at step v or at step vi; and
viii) filtering the nanoemulsion obtained at the end of step vii aseptically through 0.2μ filter, filled in vials and lyophilised.

22. A lyophilized composition prepared by a process as claimed in claim 21.

23-26. (canceled)

27. A composition as claimed in claim 3 wherein Docetaxel is 0.05%-2.0% w/v of the composition.

28. A composition as claimed in claim 3 wherein the synthetic triglyceride oil has fatty acids selected from caproic acid, caprylic acid, capric acid, lauric acid, myristic acid, oleic acid and mixtures thereof.

28. A composition as claimed in claim 3 wherein the synthetic triglyceride oil has caprylic acid 85%-100% by weight.

30. A composition as claimed in claim 3 wherein the synthetic triglyceride oil is selected from medium chain triglyceride, tricaprylin and triolein and mixtures thereof.

31. A composition as claimed in claim 3 wherein the purified natural phosphatides are selected from purified egg lecithin and purified soya lecithin and mixtures thereof.

32. A composition as claimed in claim 3 wherein the ratio by weight of synthetic triglyceride oil to Docetaxel is 1:1-100:1.

33. A composition as claimed in claim 3 wherein ratio by weight of synthetic triglyceride oil to Docetaxel is 10:1-50:1.

34. A composition as claimed in claim 4 wherein the ratio by weight of synthetic triglyceride oil to N-(carbonyl-methoxypolyethylene glycol 2000)-1,2-distearoyl-sn-glycero-3-phosphoethanolamine is 1:1-100:1.

35. A composition as claimed in claim 4 wherein ratio by weight of synthetic triglyceride oil to N-(carbonyl-methoxypolyethylene glycol 2000)-1,2-distearoyl-sn-glycero-3-pphosphoethanolamine is 5:1-20:1.

36. A composition as claimed in claim 3 wherein the ratio by weight of synthetic triglyceride oil to purified natural phosphatide is 4:1-40:1.

37. A composition as claimed in claim 3 wherein the ratio by weight of synthetic triglyceride oil to purified natural phosphatide is 7:1-20:1.

38. A composition as claimed in claim 3 wherein the glycerol content is 0.5-3% w/v of the composition.
